(19) United States
(12) Patent Application Publication
(10) Pub. No.: US 2010/0332254 A1
(43) Pub. Date: Dec. 30, 2010

(54) IN-VITRO DEVICE SUPPORT FOR X-RAY BASED KIDNEY FUNCTION TEST

(76) Inventors: Michael Maschke, Lonnerstadt (DE); Sebastian Schmidt, Weisendorf (DE); Gudrun Zahlmann, Neumarkt (DE)

Correspondence Address: BRINKS HOFER GILSON & LIONE, P.O. BOX 10395, CHICAGO, IL, 60610, US

(21) Appl. No.: 12/492,329

(22) Filed: Jun. 26, 2009

(51) Int. Cl. G06Q 50/00; A61B 5/00; G06N 5/02; A61B 6/00

(52) U.S. Cl. 705/3; 600/300; 706/52; 600/431

(57) ABSTRACT

A system and method of diagnosing kidney syndromes using an intravenous pyelogram (IVP) is described. The system includes an X-ray imaging device and an in-vitro diagnostic (IVD) device. The IVD device is used to identify risk factors such as sensitivity to contrast agents, or to indicate or rule out other syndromes which may have symptoms similar to kidney malfunction. The level of blood components such as creatinine may be used, in conjunction with other medical data and IVD tests, to determine a risk score for the patient. The risk score may be used to recommend modifications to a baseline procedure such as change in the volume or type of contrast agent, the number of X-rays, or the performance of post-procedure dialysis.

### TECHNICAL FIELD

The present application generally relates to the use of in-vitro testing to improve an X-ray based kidney function test.

### BACKGROUND

Intravenous pyelograms (IVP) are common X-ray procedures. The IVP is an X-ray examination of the kidneys, ureters and urinary bladder using a contrast material. When a contrast material is injected into a vein, the material travels through the blood stream and collects in the kidneys and urinary tract, increasing X-ray absorption in these areas. A series of images are taken at timed intervals. The kidneys are responsible for removing contrast dye from the blood and collecting it in the urine for excretion from the body.

IVP examination allows the radiologist to view and assess the anatomy and the functioning of the kidneys and lower urinary tract. The examination helps the physician assess abnormalities in the urinary system, as well as how quickly and efficiently the patient's system is able to eliminate bodily waste. The IVP may be used to help diagnose the cause of symptoms such as blood in the urine or pain in the side or lower back.

Examples of syndromes which may be diagnosed with the aid of IVP are kidney stones, an enlarged prostate, and tumors in the kidney, ureters, or urinary bladder. An advantage of using IVP is that it is a fast and cost effective means of using conventional X-ray equipment. However, when performing an IVP examination, the patient is administered a large amount of iodine contrast agents. Many patients are allergic to, or develop an allergy to, this type of contrast material and the contrast agent also requires good kidney function to be promptly eliminated from the human body. Good kidney function, however, may not be an attribute of the patient needing such an examination.

The publication, “Acute Reactions to Urographic Contrast Media”, P. Davies, M. B. Roberts, J. Roylance, British Medical Journal, 1975, 2, 434-437, describes the above mentioned problems in more detail.

WO 2005/087272, “Low Osmolar X-ray Contrast Media Formulations” discloses contrast agents for pyelography.

Alternative examination protocols could be examinations using computed tomography (CT) or magnetic resonance imaging (MRI); however, these examinations are much more expensive. Moreover the CT protocol results in the patient being exposed to a higher dose of radiation.

In-vitro diagnostic (IVD) products are those reagents, instruments, and systems intended for use in diagnosis of disease or other conditions, including a determination of the state of health, in order to cure, mitigate, treat, or prevent disease or its sequelae. Such IVD products are intended for use in the collection, preparation, and examination of specimens taken from the human body.

An example of an IVD device is a blood analysis device such as “Lab on a Chip”, which is being developed by Siemens A G, may be used for determining further blood chemistry values or to identify genetic or molecular markers (see, for example, WO 00/56922, “Genetic Polymorphism and Polymorphic Pattern for Assessing Disease Status, and Compositions for Use Thereof”, and WO 00/23802, “Method for_Measuring Cellular Adhesion” for gene tests and tests with molecular markers for stroke). See also, WO 2005/106024, “Method and Assembly for DNA Isolation with Dry Reagents” and WO 2005/106023, “PCR Process and Arrangement for DNA Amplification using Dry Reagents.” All of the above references are incorporated herein by reference.

### SUMMARY

A method of performing a diagnostic procedure on a patient is disclosed, including, selecting a diagnostic procedure; retrieving existing patient medical records; and, determining whether appropriate in-vitro diagnostic tests (IVD) have been performed. Where needed IVD tests have not been performed the tests are ordered or performed and the data is received. Using the patient records and IVD test results, a patient risk score is determined, and the procedure steps and parameters are selected based on the patient risk score.

In an aspect, a computer program product, stored on a computer readable medium, includes instructions for configuring a computer to request medical data of a patient from a data base; receive the medical data from a data base through an interface to a network; and accept an operator input identifying the diagnostic procedure to be planned. A set of IVD test data needed for planning the procedure is determined and, if all of the required results are not available, additional tests are requested, and the results of the tests received. A patient risk score is determined for the diagnostic procedure, and changes to the diagnostic procedure recommended based on the risk score.

In another aspect, a system for performing an intravenous pyelogram (IVP) includes an X-ray device, and an in-vitro diagnostic device (IVD). The IVD is used to perform a test for at least one syndrome related to patient symptoms, and at least one of a type or volume of contrast agent, or a number of X-rays during the IVP is changed from a low-risk baseline procedure.

### BRIEF DESCRIPTION OF THE DRAWINGS

FIG. 1 is a block diagram illustrating a diagnosis system; and

FIG. 2 is a block diagram illustrating a method of planning a diagnosis procedure.

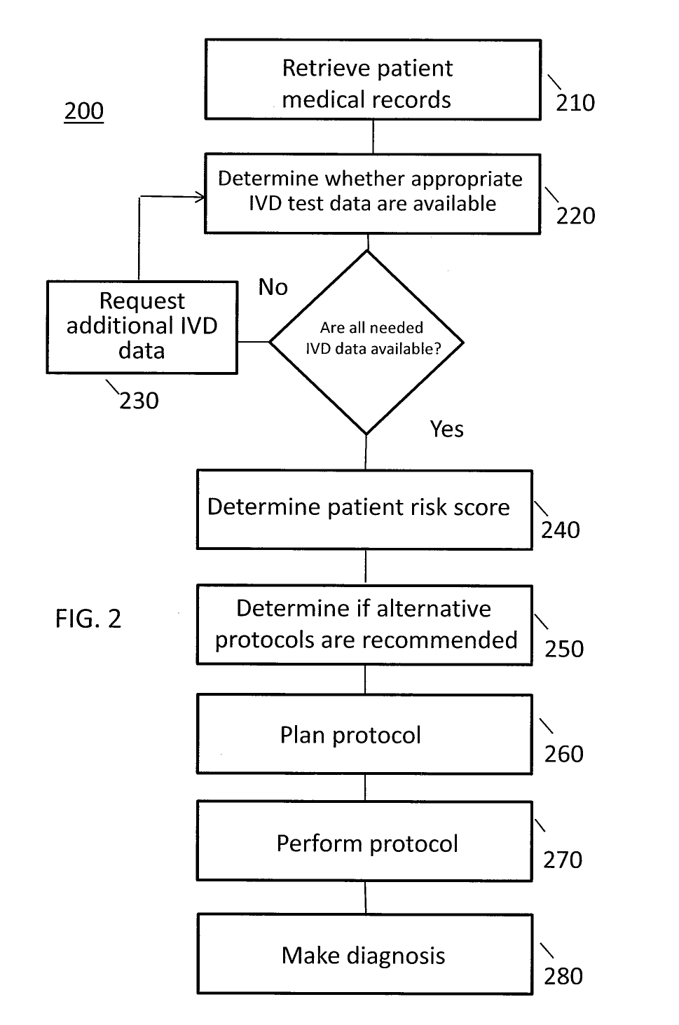

### DESCRIPTION

Exemplary embodiments may be better understood with reference to the drawings, but these embodiments are not intended to be of a limiting nature.

The combination of hardware and software to accomplish the tasks described herein may be termed a platform, treatment suite, system, or the like. The instructions for implementing processes of the platform may be provided on computer-readable storage media or memories, such as a cache, buffer, RAM, FLASH, removable media, hard drive or other computer readable storage media. Computer readable storage media include various types of volatile and nonvolatile storage media. The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks described herein may be performed by dedicated hardware, or manually by an operator.

In an embodiment, the instructions may be stored on a removable media device for reading by local or remote systems. In other embodiments, the instructions may be stored in a remote location for transfer through a computer network, a local or wide area network, by wireless techniques, or over telephone lines. In yet other embodiments, the instructions are stored within a particular computer, system, or device.

Where the term “data network”, “web” or “Internet”, or the like, is used, the intent is to describe an internetworking environment, which may include both local and wide area telecommunications networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths. Often, the internetworking environment is provided, in whole or in part, as an attribute of the facility in which the platform is located.

Communications between the devices, systems and applications may be by the use of either wired or wireless connections. Wireless communication may include, audio, radio, lightwave or other technique not requiring a physical connection between a transmitting device and a corresponding receiving device. While the communication may be described as being from a transmitter to a receiver, this does not exclude the reverse path, and a wireless communications device may include both transmitting and receiving functions. Such wireless communication may be performed by electronic devices capable of modulating data as a signal on a carrier wave for transmission, and receiving and demodulating such signals to recover the data. The devices may be compatible with an industry standard protocol such as IEEE 802.11b/g, or other protocols that exist, or may be developed.

The terms used herein are believed to be, and are meant to be interpreted as, understood by a person of skill in the art at the time of preparation of the specification, unless specifically differentiated herein.

When describing a medical intervention technique, the terms “non-invasive,” “minimally invasive,” and “invasive” may be used. Generally, the term non-invasive means the administering of a treatment or medication while not introducing any treatment apparatus into the vascular system or opening a bodily cavity. Included in this definition is the administering of substances such as contrast agents using a needle or port into the vascular system. Minimally invasive means the administering of treatment or medication by introducing a device or apparatus through a small aperture in the skin into the vascular or related bodily structures. Invasive techniques may include conventional surgery.

The disadvantages to the patient of the IVP examination may be mitigated by the use an IVD test in combination therewith. The IVD may be used as an additional indicator leading to the proper diagnosis. By using the IVD test the concentration of the contrast media may be reduced, or the X-ray dose may be reduced.

In an aspect, IVD tests which may be used effectively are those which may substantially rule out or suggest specific syndromes as a diagnosis may be employed, such as: U.S. Pat. No. 7,196,063, “Peptides and Pharmaceutical Compositions Comprising Same”, which discloses a marker which indicates the potential of kidney stones; WO 2006/056766, “Diagnosis of Prostate Cancer” which discloses a marker to indicate an enlarged prostate; and, WO 2006/084075, “Adam-9 Modulators,” which discloses an antigen and antibody test to indicate tumors in the kidney, ureters, or urinary bladder.

In another aspect the IVD test results may permit the reduction of the amount of contrast media (for example, by thinning the contrast solution with a NaCl solution, eliminating the use of contrast media, or reducing the amount of the X-ray dose.

In yet another example, the image sample rate in the IVP may reduced by performing the X-ray imaging studies a fewer number of times at an increased interval. In an aspect, a dynamic IVD test based measuring procedure may be used where at the point of interest (kidney, ureters, and urinary bladder) the incoming contrast media concentration is measured. At the epoch of peak inflow, or any other appropriate time point, the X-ray imaging procedure may be initiated.

The choice of contrast agent may also be influenced by the likelihood of an adverse patient reaction, such as that to iodine-based contrast agents. An IVD test for such sensitivity may be performed as part of the safety measures related to planning the IVP. This is further described in U.S. Ser. No. ______, filed on Jun. 26, 2009, “System And Method For To Reducing Patient Risk Of Allergic Reaction To Contrast Agents Or Medical Material.” This application is commonly owned and is incorporated by reference herein.

A method of use of the IVD device in combination with the X-ray system for diagnosis may include the following steps: for an identified patient, the relevant prior IVD test data and other tests may be retrieved from a hospital data base; other IVD tests may be initiated if the relevant tests have not been performed or, if the results of the previous tests suggest additional testing. The IVD test results and patient data, including biometric data, maybe used to calculate a risk of adverse effects of the IVP procedure, which may be expressed quantitatively as a risk score, and adjusting the imaging parameters, including adjusting the dosage of contrast material, or otherwise guide the workflow.

An example of a risk score is one which is a measure of the risk of contrast-material-induced kidney disease. The risk score computation may include the use of blood creatinine level, blood urea, and patient age; medication such as ACE-inhibitors or diuretics; hypertension; and known NSAID abuse, or other substance abuse.

If computed risk is low (e.g., creatinine <1.2 mg/dl, and no other risk factors) a normal protocol is chosen. If risk is medium (e.g., 4> creatinine >1.2 mg/dl, or more than two other risk factors), the time between the images may be increased: for example, doubled, and a reduced volume of contrast agent is recommended. Alternatively, the system can recommend the use of a special contrast agent (CA), such as, isoosmolar agents. If the risk is very high (e.g. creatinine >4), the system may recommend the use of another imaging procedure or additional measures, such as renal dialysis, after the examination. Pre-examination treatments may also be indicated to mitigate patient reaction to the procedure.

Where all of the required IVD data cannot be obtained prior to the procedure, other medical information may be used to assess the risk on a qualitative basis, where the severity of the patient consequences is taken into account where a numerical value is used.

In another aspect, the obtained images may be analyzed and the approximate amount of contrast agent eliminated by the kidneys during the examination may be estimated. This estimate can be performed by subtracting an image obtained from prior to the CA injection, and calculating an integral of the X-ray absorption over the area of interest. Generally, the CA material is excreted from the kidney to the bladder during the course of the examination, and thus the retention of CA material can be estimated. If the results do not indicate an appropriate amount of excretion, the initiation of dialysis may be indicated.

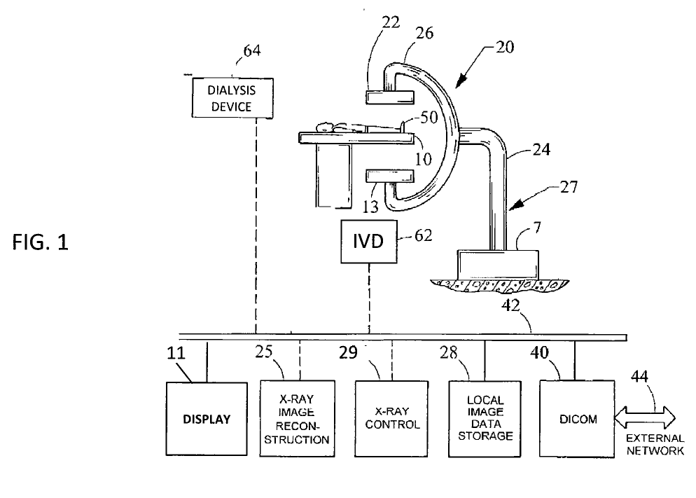

FIG. 1 shows a block diagram of an example of a system for using an IVD device to complement an IVP procedure to perform a minimally invasive diagnosis procedure. Other embodiments of the system may include fewer than all of the devices, or functions, shown in FIG. 1. It will be understood by persons of skill in the art that the signal and data processing and system control is shown in an example, and that many other physical and logical arrangements of components such as computers, signal processors, memories, displays and user interfaces are equally possible to perform the same or similar functions. The particular arrangement shown is convenient for explaining the relationship of the elements and the functionality of the system.

A C-arm X-ray device 20 is representative of an imaging modality which may be used and comprises a C-arm support 26 to which an X-ray source 22, which may include a diaphragm to limit the field of view, and an X-ray detector 13 may be mounted so as to face each other along a central axis of radiation. The C-arm 26 is mounted to a robotic device 27 comprising a mounting device 7, and one or more arms 24 which are articulated so as to be capable of positioning the C-arm X-ray device with respect to a patient support apparatus 10.

The C-arm X-ray device 20 may be used to obtain a sequence of projection X-ray image data of a patient 50, and the images are reconstructed by any suitable technique of image data processing.

The IVD device 62 may be mobile or fixedly installed. In an example, a “point-of-care” device positioned in proximity to the patient permits a patient bodily fluid to be drawn and placed in the IVD device. US 2008/0312519 describes a mobile examination unit having an integrated set of sensors based on a “Lab-on-a-chip” such as described in DE 10 2004 021 780 and DE 10 2004 822 A mobile analyzer can serve several treatment rooms and may be moved to the one where a treatment procedure is currently being performed. Another example of a point-of-care device, which could be installed in each treatment room, as disclosed in US 2005/0123444, is desktop analyzer. Another point-of-care IVD is disclosed in U.S. Pat. No. 6,845,327 which may have analytic sensors in each treatment room to obtain the body fluid sample and perform a pre-analysis, where a final analysis may be performed on a central computer accessed over a communications network, which may be a local area network (LAN).

Alternatively, depending on the equipment available, is also possible to use the more centralized kind of body-fluid analyzers where, for example, a blood sample is drawn in a small container, which is sent down to a central lab and the results are send back by FAX or other means. Although the specific tests that would be performed by such an analyzer may differ from therapy-type-to-therapy-type, a person of skill in the art would be capable of selecting an appropriate analyzer or request the development of an analyzer suitable for obtaining the required data.

The devices and functions shown in FIG. 1 are representative, but not inclusive. Other imaging modalities such as MRI, CT, US and the like, either individually or in combination. The individual units, devices, or functions may communicate with each other over cables or in a wireless manner, and the use of dashed lines of different types for some of the data and control connections in is intended to suggest that alternative means of connectivity may be used.

Images reconstructed from the X-ray data may be stored in a non-volatile (persistent) storage device 28 for further use. The X-ray device 20 and the image processing attendant thereto may be controlled by a separate controller 29 or the function may be consolidated with the user interface and display 11, or performed by a separate image processor 25.

The various devices may communicate with a DICOM (Digital Communication in Medicine) system 40 over a local area network 42 and with external devices over a network interface 44.

Some or all of the signal and data processing and data display may also be located in the treatment room; however, equipment and functionality not directly related to the sensing or manipulating of the patient or the interventional device, may be remotely located. Such remote location may be facilitated by high speed data communications on local area networks, wide area networks, and the Internet. The signals representing the data and images may be transmitted by modulation of representations of the data on electromagnetic signals such as light waves, radio waves, or signals propagating on wired connections.

The system sensors, such as the IVD device 62, the X-ray device 20 and the dialysis device 64, may thus be located remotely from the specialists making the diagnosis and for determining or administering the appropriate course of treatment. Of course, the specialists may be present with the patient at times as well.

The data processing and system control is shown as an example, and many other physical and logical arrangements of components such as computers, signal processors, memories, displays and user interfaces are equally possible to perform the same or similar functions. The particular arrangement shown is convenient for explaining the functionality of the system.

The system of FIG. 1 may be used to perform a method (200) of diagnosis of a patient syndrome, the method including the steps of: retrieving patient medical history records (step 210); determining whether appropriate preparatory IVD test results are available for use in planning the diagnostic protocol (step 220); performing diagnostic tests that may not have been performed (step 230); determining a risk measure to the patient in performing the diagnostic protocol (step 240); determining if alternative protocols or parametric values are recommended (step 250); planning the protocol based on the risk measure and other medical and economic factors, including the availability of equipment and personnel (step 260); performing the planned diagnostic protocol (step 270); and interpreting the results of the protocol (step 280) (make diagnosis).

In an aspect, when performing step 220 when planning a procedure in the protocol where a contrast material is to be used, a step of performing an IVD test to determine whether a sensitivity to iodine-based materials is present is performed.

In another aspect, when performing step 240, the level of kidney function may be assessed by blood characteristic analysis, and the probability of specific syndromes which may be responsible for the patient symptoms determined using IVD diagnosis, so the aspects of the protocol using X-ray exposure, and contrast agents may be performed at less risk to the patient. In the case of IVP, for example, the blood creatinine level may be used as an important risk measure. This may result in a recommendation for additional pre-procedure or post-procedure measures to minimize the effect on the patient. The risk level may be expressed numerically, or by grades such as low, medium or high.

The examples of diseases, syndromes, conditions, and the like, and the types of examination and treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

Apart from the sensors positioning and catheterization capabilities, the imaging, data processing, and controlling equipment may be located within the treatment room or remotely, and the remotely-located equipment may be connected to the treatment room by a telecommunications network. Aspects of the diagnosis and treatment may be performed without personnel, except for the patient, being present in any of the local treatment rooms.

It is intended that the foregoing description be regarded as illustrative rather than limiting, and that it be understood that it is the following claims, including all equivalents, that are intended to define the spirit and scope of this invention.

What is claimed is:

1. A method of performing a diagnostic procedure on a patient, the method comprising:
selecting a diagnostic procedure;
retrieving existing patient medical records;
determining whether appropriate in-vitro diagnostic tests (IVD) have been performed;
performing needed IVD tests;
determining a risk score for the patient; and
selecting procedure steps and parameters based on the patient risk score.

2. The method of claim 1, wherein the diagnostic procedure is an intravenous pyelogram (IVP).

3. The method of claim 2, wherein a factor in determining the risk score is blood creatinine level as measured by an IVD.

4. The method of claim 1, wherein the needed IVD tests include at least one of an IVD test for diagnosis of: kidney stones, an enlarged prostate, or tumor in the kidney, ureters, or urinary bladder.

5. The method of claim 4, wherein the needed IVD tests include a test for iodine sensitivity.

6. The method of claim 1, wherein the risk score is used to modify at least one of a volume of contrast agent administered, or a period between X-ray exposures.

7. The method of claim 1, wherein when the risk score is very high, a dialysis procedure is performed on the patient after completion of the diagnostic procedure.

8. A computer program product, stored on a computer readable medium, comprising:
instructions for configuring a computer to:
request medical data of a patient from a data base;
receive the medical data from a data base through an interface to a network;
accept an operator input identifying the diagnostic procedure to be planned;
determine a set of IVD tests needed for the procedure, and whether all of the needed tests have are present in the medical data;
request any needed IVD tests that are not present in the medical data and accept the results of the requested IVD tests;
determine a risk score to the patient for the diagnostic procedure; and
recommend changes to the diagnostic procedure based on the risk score.

9. The computer program product of claim 8, wherein the risk score includes at least one of the following factors: a probability that a syndrome causing patient symptoms has been diagnosed by the performed IVD tests, and a sensitivity of the patient to the contrast agent as determined from one of the received medical data or the performed IVD tests.

10. The computer program product of claim 9, wherein the diagnostic procedure is an intravenous pyelogram (IVP).

11. The computer program product of claim 10, wherein the risk score includes the blood creatinine level as a factor.

12. The computer program product of claim 11, wherein when the creatinine level is very high a post-IVD dialysis procedure is recommended.

13. The computer program product of claim 11, wherein a volume of contrast agent or a number of X-ray exposures is adjusted based on the risk score.

14. A system for performing an intravenous pyelogram (IVP), comprising:
an X-ray device; and
an in-vitro diagnostic device (IVD),
wherein the IVD is used to perform a test for at least one syndrome related to a patient symptoms, and at least one of a type or volume of contrast agent, or a number of X-rays during the IVP is changed from a low risk baseline procedure.

15. The system of claim 14, wherein the IVD is a point-of-service device.

16. The system of claim 14, wherein the IVD is used to perform a test for iodine sensitivity.

17. The system of claim 14, wherein the IVD is used to perform a test for blood creatinine level.

18. The system of claim 17, wherein when the blood creatinine level is very high, the system further comprises a kidney dialysis device.
